(19) United States
(12) Patent Application Publication
(10) Pub. No.: US 2019/0033186 A1
(43) Pub. Date: Jan. 31, 2019

(54) METHOD FOR CAPTURING RARE CELLS

(71) Applicant: HITACHI CHEMICAL COMPANY, LTD., Tokyo (JP)

(72) Inventors: Seita NAKAMURA, Chiyoda-ku, Tokyo (JP); Katsuya ENDOU, Chiyoda-ku, Tokyo (JP); Masayuki HIGUCHI, Chiyoda-ku, Tokyo (JP); Hisashige KANBARA, Chiyoda-ku, Tokyo (JP)

(21) Appl. No.: 16/071,988

(22) PCT Filed: Dec. 1, 2016
(86) PCT No.: PCT/JP2016/085775

§ 371 Date: Jul. 23, 2018

(30) Foreign Application Priority Data
Jan. 25, 2016 (JP) 2016-011427

(51) Int. Cl. G01N 1/40; G01N 33/574

(52) U.S. Cl. CPC G01N 1/4077; G01N 2001/4088; G01N 33/574

(57) ABSTRACT

The present invention provides a method for capturing rare cells, comprising a step of adding beads to a fluid containing rare cells and contaminating cells; and a step of filtering, with a filter, the fluid to which the beads have been added. According to this method, rare cells can be enriched with the rare cells selectively captured from a fluid containing the rare cells and contaminating cells.

### TECHNICAL FIELD

The present invention relates to a method for capturing rare cells.

### BACKGROUND ART

Research and clinical significance of cancer cell enrichment are extremely great, and if cancer cells in blood can be enriched, namely, if cancer cells can be selectively captured, it can be applied to diagnosis of cancer. For example, the most significant element in prognosis and treatment of cancer is whether or not cancer cells have metastasized at the time of the first medical consultation or the treatment. When cancer cells are diffused to peripheral blood at an initial stage, detection of circulating tumor cell (hereinafter also referred to as a “CTC”) is useful means for determining the progression of cancer. It is, however, difficult to detect a CTC present in an extremely small amount because there exist, in blood, a predominantly large amount of blood components such as an erythrocyte and a leukocyte. Circulating endothelial cell (hereinafter also referred to as a “CEC”) is also used as a biomarker for the prognosis and the treatment of cancer, but CEC exists in an extremely small amount in blood, and hence is difficult to detect similarly to the CTC.

An example of a method for enriching rare cells such as the CTC and the CEC from blood includes a method using a filter (hereinafter also referred to as a “filtering method”). The filtering method is a method in which rare cells are enriched utilizing a difference in the size and the deformability of cells.

As an example of the filtering method, a method for detecting a small amount of the CTC by utilizing a resin filter of parylene has been proposed (Patent Literature 1).

As another example of the filtering method, a method in which leukocytes and cancer cells are separated from each other depending on a difference in the deformability therebetween by using a filter of a metal to improve the strength of the filter has been proposed (Patent Literatures 2 to 4).

### CITATION LIST

### Patent Literature

Patent Literature 1: International Publication No. WO2010/135603

Patent Literature 2: Japanese Unexamined Patent Publication No. 2013-42689

Patent Literature 3: Japanese Unexamined Patent Publication No. 2011-163830

Patent Literature 4: International Publication No. WO2015/012315

### SUMMARY OF INVENTION

### Technical Problem

In recent years, owing to improvement in technologies of a next generation sequencer capable of analyzing genes from a small number of cells, digital PCR and quantitative reverse transcription PCR (qRT-PCR), research has been shifted to “qualitative assessment” for detecting gene mutation and protein expression level of rare cells. This is because a screening of novel marker proteins and genes using the results of the genetic analysis of rare cells is desired.

When the genetic analysis is performed under condition where contaminating cells typified by leukocytes exist, genetic information of rare cells may be buried in genetic information of the contaminating cells, and hence the genetic information of the rare cells may not be detected or a false negative result may be obtained in some cases. In the filtering method described in Patent Literatures 1 to 4, however, the purity of separated rare cells is not sufficiently high for the genetic analysis or the like.

The present invention was devised in consideration of these problems, and provides a method for enriching rare cells with the rare cells selectively captured from a fluid containing the rare cells and contaminating cells.

### Solution to Problem

As a result of earnest studies, the present inventors have found that rare cells can be selectively captured and enriched by filtering, with a filter, a fluid containing the rare cells and contaminating cells in the presence of beads, and thus, the present invention was accomplished.

A method for capturing rare cells of the present invention comprises a step of adding beads to a fluid containing rare cells and contaminating cells; and a step of filtering, with a filter, the fluid to which the beads have been added. Although not restricted by a specific theory, the present inventors speculate the function of the beads in the present invention as follows: The contaminating cells remaining on the filter pile up in layers and clog pores of the filter, and thus, filtration efficiency is lowered. The beads push out the layered contaminating cells to unclog the filter and prevent the filtration efficiency from lowering, and as a result, the rare cells can be selectively captured.

A median diameter of the beads can be 0.5 μm to 8 μm.

An amount of the beads added can be 1×10⁴ to 9×10⁶ beads per 1 mL of the fluid.

The beads can be at least one type of resin beads and magnetic beads.

The contaminating cells can be leukocytes.

The rare cells can be circulating tumor cells or circulating endothelial cells.

### Advantageous Effect of Invention

According to the present invention, rare cells can be enriched with the rare cells selectively captured from a fluid containing the rare cells and contaminating cells.

### BRIEF DESCRIPTION OF DRAWINGS

FIG. 1 is a schematic diagram of a filter.

FIG. 2 is a schematic diagram illustrating a structure of a CTC capturing apparatus.

### DESCRIPTION OF EMBODIMENT

The term “rare cells” refers to cells to be captured, and means cells present in an extremely small ratio with respect to a total number of all the cells contained in a fluid containing the rare cells and contaminating cells. The rare cells are, for example, cancer cells such as CTCs or endothelial cells such as CECs.

The term “contaminating cells” refers to cells excluding the rare cells contained in the fluid containing the rare cells and the contaminating cells. The number of contaminating cells is usually extremely large as compared with the number of the rare cells. Herein, the “contaminating cells” may refer to cells, among such cells, having a similar diameter to the rare cells and having deformability in some cases, and more specifically, may refer to leukocytes in some cases.

Here, the “diameter” of a cell refers to a length of the longest line segment among line segments connecting arbitrary two points on the contour of the cell observed with a microscope.

The fluid containing the rare cells and the contaminating cells can be, for example, blood, ascitic fluid or pleural fluid.

The term “capture” means causing cells to remain on a filter by filtering a fluid containing the cells with the filter. The term “selectively capture” means that a ratio of prescribed cells in a cell population remaining on the filter is higher than a ratio thereof in the fluid containing the cells before the filtration.

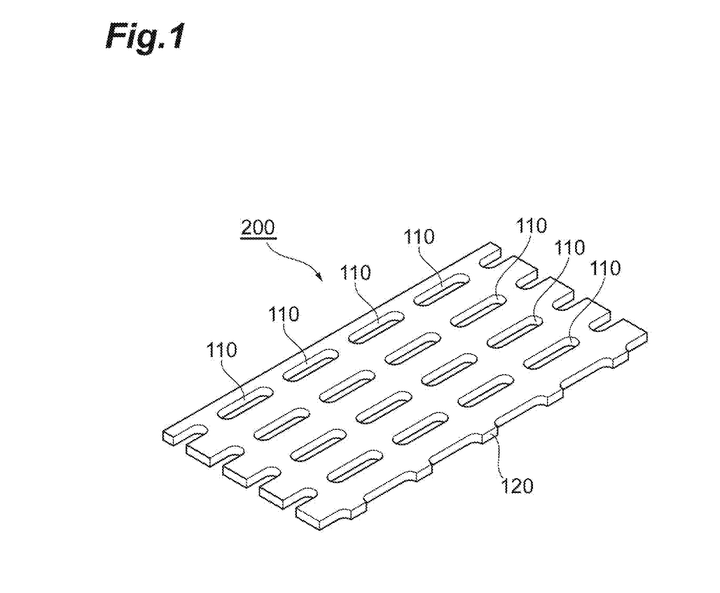

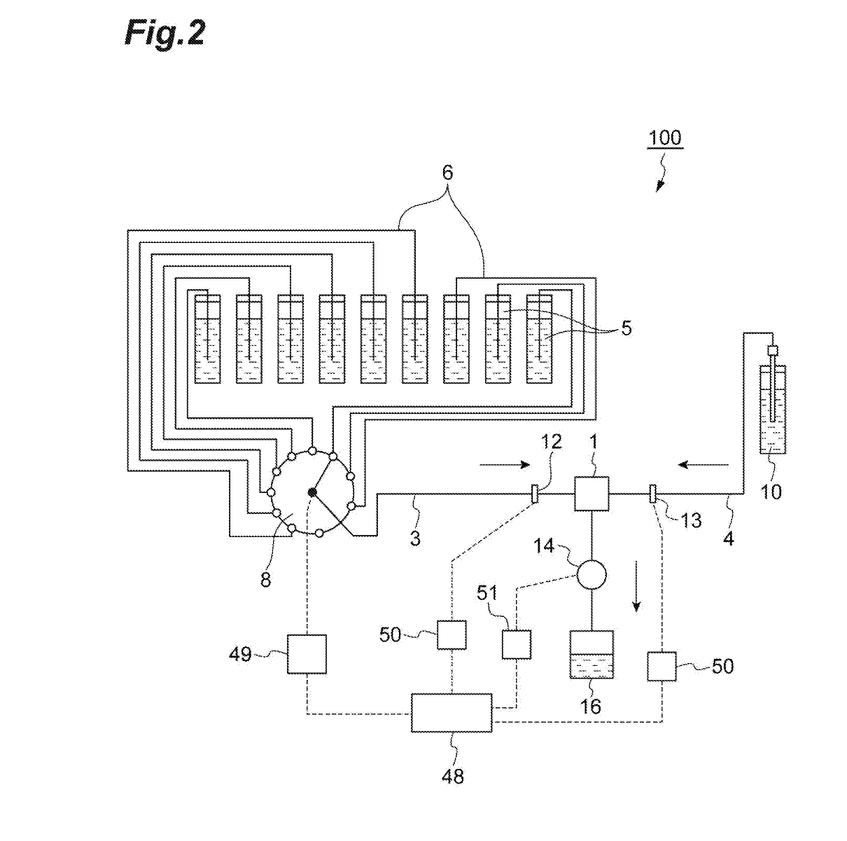

A method for capturing rare cells of the present invention includes a step of adding beads to a fluid containing rare cells and contaminating cells; and a step of filtering, with a filter, the fluid to which the beads have been added. Now, these steps will be described in detail with reference to FIG. 1 and FIG. 2.

First, the step of adding beads to a fluid containing rare cells and contaminating cells will be described.

The shape of the beads is not especially limited, and can be in the shape of, for example, a sphere, a rectangular parallelepiped, a triangular pyramid or a cone. From the viewpoint of reducing local impact caused when the beads collide against the rare cells and minimizing damage of rare cells, the beads are preferably in the shape of a substantial sphere or a sphere, and are more preferably in the shape of a sphere.

A median diameter of the beads can be appropriately selected in accordance with the dimeters of the rare cells and the contaminating cells and a pore size of the filter. For example, when the rare cells are CTCs or CECs and the contaminating cells are leukocytes, the median diameter of the beads is preferably 0.5 μm to 8 μm, more preferably 1 μm to 7.5 μm, and further preferably 3 μm to 7 μm from the viewpoints of preventing the filter from clogging, pushing out the leukocytes, and observing the captured rare cells. Here, the median diameter of the beads refers to a particle size at which a cumulative value in a particle size distribution measured with a laser diffraction particle size distribution analyzer is 50%.

The material of the beads is not especially limited, and can be, for example, resins (polystyrene, polymethacrylate, polylactic acid and the like), metals or metal oxides (iron oxide and the like), polysaccharides (chitosan and the like), silica or a combination of any of these. Among these, resin beads are preferred.

It is preferable that the beads be magnetic beads. Magnetic beads are beads containing a magnetic substance. The magnetic beads may, for example, be a magnetic substance coated with a resin, a metal or a metal oxide, a polysaccharide, silica or a combination of any of these.

The surface of the beads can be coated with a material having an effect of suppressing adsorption of, for example, DNA, RNA and protein. Examples of such a coating material include polyethylene glycol (PEG), bovine serum albumin (BSA), parylene and a 2-methacryloyloxyethyl phosphorylcholine monomer (MPC monomer).

An amount of the beads added is preferably 1×10⁴ to 9×10⁶, more preferably 1×10⁵ to 7×10⁶, and further preferably 5×10⁵ to 5×10⁶ beads per 1 mL of the fluid containing the rare cells and the contaminating cells. When the amount of the beads added is equal to or larger than the lower limit, the contaminating cells can be more effectively pushed out. When the amount of the beads is equal to or smaller than the upper limit, the beads do not clog the filter, and the captured rare cells can be more easily observed. Besides, the amount of the beads added is appropriately adjusted in accordance with the number of cells contained in the fluid containing the rare cells and the contaminating cells. The amount of the beads added is preferably 0.002 to 100 times, more preferably 0.02 to 75 times, and further preferably 0.2 to 50 times as large as the number of the contaminating cells contained in the fluid containing the rare cells and the contaminating cells. When the amount of the beads added falls in the above-described numerical range, the contaminating cells can be more effectively pushed out.

When the fluid containing the rare cells and the contaminating cells is blood, a blood sample can be obtained by collecting blood from a subject by a usual method. In this case, an additive conventionally added to a blood sample, such as an anticoagulant or a fixative, can be further added thereto. Examples of the anticoagulant include ethylenediamine tetraacetic acid disodium salt dihydrate (EDTA-2Na), ethylenediamine tetraacetic acid dipotassium salt dihydrate (EDTA-2K), sodium citrate, sodium fluoride, heparin and a CTAD solution (a mixed solution of citric acid, theophylline, adenosine and dipyridamole). Examples of the fixative include paraformaldehyde (PFA), glutaraldehyde and ethanol. Such an additive may be precedently put in a blood collecting tube to be used for blood collection, or may be added thereto after the blood collection. The blood sample may be diluted with, for example, a buffer.

Next, the step of filtering, with a filter, the fluid containing the rare cells and the contaminating cells to which the beads have been added will be described.

Through this step, the contaminating cells are removed from the fluid containing the rare cells and the contaminating cells to selectively capture the rare cells.

Now, the structure of a filter usable in this step will be described with reference to FIG. 1. A filter 200 has a structure in which a large number of through holes 110 are formed in a thin film 120 of a plastic, a metal or the like.

The shape of an opening of the through hole 110 can be, for example, a circle, an ellipse, a square, a rectangle, a round rectangle, or a polygon. From the viewpoint of efficiently capturing the rare cells, the shape of the opening is preferably a circle, a rectangle or a round rectangle, and more preferably a rectangle or a round rectangle. A round rectangle refers to a shape having a rectangle and two semicircles each having a radius equal to the length of a short side of the rectangle and each adjacent to and each attached to one of the two short sides of the rectangle. When the shape of the opening is a rectangle or a round rectangle, the through holes 110 are unlikely to clog and the rare cells can be more selectively captured. The through holes 110 may be in an aligned arrangement as illustrated in FIG. 1, may be in a staggered arrangement in which their positions are shifted every row, or may be in a random arrangement in which they are arbitrarily arranged.

A pore size of the through hole 110 is set depending on the diameter of the rare cells to be captured. When the rare cells have a diameter of 10 μm or more like a CTC, the pore size of the through hole 110 is preferably 5 μm to 15 μm, more preferably 6 μm to 12 μm, and further preferably 7 μm to 10 μm. It is noted that the pore size of the through hole 110 (sometimes simply referred to as the “pore size of the filter”) herein refers to the maximum value of a diameter of a sphere capable of passing through the through hole 110. For example, when the shape of the opening is a rectangle, the pore size of the through hole 110 corresponds to the length of the short side of the rectangle. When the shape of the opening is a polygon, the pore size of the through hole 110 corresponds to a diameter of an inscribed circle of the polygon. In the case where the shape of the opening is a rectangle or a round rectangle, there remains a gap in the lengthwise direction of the opening shape in the through hole 110 even when the rare cell is captured in the through hole 110. Since the contaminating cells can pass through this gap, the filter 200 can be prevented from clogging.

Open area ratio of the filter 200 is preferably 5 to 50%, more preferably 10 to 40%, and further preferably 10 to 30% from the viewpoint of keeping balance between the strength of the filter 200 and the prevention of the clogging of the filter 200. It is noted that the open area ratio herein refers to an area occupied by the through holes 110 in the total area of the filter 200.

A thickness of the filter 200 is preferably 3 μm to 50 μm, more preferably 5 μm to 40 μm, and further preferably 5 μm to 30 μm from the viewpoint of keeping the balance between the strength of the filter 200 and the prevention of the clogging of the filter 200.

It is preferable that the filter 200 be made of a metal. Since a metal is excellent in processability, the processing accuracy of the filter 200 can be thus improved, so as to further improve a ratio of capturing the rare cells. Besides, since a metal is more rigid than other materials, the size and the shape are retained even when an external force is applied. Therefore, even when the contaminating cells are rather larger than the through hole 110, the filter 200 is not deformed but the contaminating cells is deformed to pass through the through hole 110, and thus, the rare cells can be more selectively captured.

Examples of the metal include nickel, silver, palladium, copper, iridium, ruthenium, chromium and an alloy of any of these. Palladium and iridium have a high oxidation-reduction potential, are poorly soluble and have good characteristics, but are expensive. Nickel has a lower oxidation-reduction potential than hydrogen and hence is easily soluble, but is inexpensive. Silver and palladium are noble metals, and are more inexpensive than palladium and iridium.

A method for producing the filter 200 is not especially limited, and the filter can be produced, for example, as follows: First, a photoresist is placed on a substrate in a position where the through holes 110 of the filter are to be formed, the substrate is plated with a metal which will later be the filter 200, and thus, a metal layer (a thin film 120) having the through holes 110 is formed on the substrate.

Thereafter, the substrate and the photoresist are removed from the metal layer to obtain the filter 200. As the substrate, a substrate with copper plating on the surface can be used. Copper is easily removable through chemical dissolution using a chemical and is excellent in adhesion to a photoresist, and hence is preferable as the material of the substrate.

A method for filtering the fluid containing the rare cells and the contaminating cells and an apparatus used for the filtration are not especially limited, but a flow rate of the fluid when filtering is preferably controlled. The flow rate of the fluid when filtering is preferably 50 μL/min to 3000 μL/min, more preferably 100 μL/min to 1000 μL/min, and further preferably 200 μL/min to 600 μL/min from the viewpoints of minimizing damage of the rare cells, pushing out the contaminating cells, and preventing the clogging of the filter 200.

After capturing the rare cells, the captured rare cells are collected, and thus, a fluid containing the rare cells can be obtained. Examples of a method for collecting the rare cells include back wash, micromanipulation and pipetting. The back wash refers to obtaining cells captured on a filter by letting a fluid to flow from a surface opposite to the surface where the cells are captured. The micromanipulation refers to collection of cells one by one from a filter with a pico pipette. The pipetting refers to peeling off cells captured on a filter by mixing a fluid on the filter with a micro pipette.

### EXAMPLES

Now, the present invention will be specifically described based on examples, and it is noted that the present invention is not limited to the following examples.

(Production of Filter)

A photosensitive resin composition (PHOTEC RD-1225: thickness of 25 μm, manufactured by Hitachi Chemical Co., Ltd.) was laminated on one surface of a 250 mm square substrate (MCL-E679F: substrate obtained by laminating a peelable copper foil onto a surface of MCL, manufactured by Hitachi Chemical Co., Ltd.). The lamination was performed at a roll temperature of 90° C., a pressure of 0.3 MPa and a conveyor speed of 2.0 m/min.

A glass mask having an opening with an open area ratio of 6.7% was placed on the surface of the substrate laminated with the photosensitive resin composition. The opening of the glass mask corresponds to a light transmitting portion, and through holes each in a round rectangular shape with a size of 7.8 μm×100 μm were aligned at a constant pitch along long axis and short axis directions of the glass mask to extend in the same direction. It is noted that the size of the round rectangle is defined by a length of a short side (7.8 μm) and a length of a long side (100 μm) of a rectangle that constitute the round rectangle.

Under vacuum of 600 mmHg or less, the substrate on which the glass mask was placed was irradiated with ultraviolet by using a UV irradiation apparatus at an exposure of 30 mJ/cm².

After the UV irradiation, the substrate was developed with a 1.0% sodium carbonate aqueous solution, so as to form on the substrate, a resist layer in which round rectangular photoresists were aligned. The substrate with the resist layer formed thereon was treated with a nickel plating solution of pH 4.5 at a solution temperature of 55° C. for about 20 minutes, and thus, nickel plating with a thickness of about 20 μm was formed on an exposed portion of the copper foil. The composition of the nickel plating solution is shown in Table 1.

TABLE 1

| Composition of Plating Liquid | Concentration (g/L) |
|---|---|
| Nickel sulfamate | 450 |
| Nickel chloride | 5 |
| Boric acid | 30 |

The thus obtained nickel plating layer was peeled off from the substrate together with the peelable copper foil. The resultant copper foil having the nickel plating thereon was stirred in an etchant (Mec Bright SF-5420B, manufactured by MEC Co., Ltd.) at a temperature of 40° C. for about 120 minutes to remove the copper foil portion through chemical dissolution, and thus, a self-supported film of nickel (in a size of 20 mm×20 mm) to be used as a metal filter was obtained.

The self-supported film was subjected to an ultrasonic treatment for about 40 minutes in a resist stripping agent (P3 Poleve, manufactured by Henkel) at a temperature of 60° C. to remove the photoresist remaining in the self-supported film, and thus, a metal filter having fine through holes was obtained. The completed metal filter had no damage of wrinkle, bend, scratch or curl, and had through holes with sufficient accuracy.

The metal filter was immersed in an acidic degreasing agent, Z-200 (manufactured by World Metal Co., Ltd), at a temperature of 40° C. for 3 minutes to remove an organic substance remaining on the metal filter.

After washing the metal filter with water, the resultant metal filter was immersed in a fluid obtained by excluding gold sulfite, that is, a gold source, from a non-cyanogen-based electroless displacement gold plating solution, HGS-100 (manufactured by Hitachi Chemical Co., Ltd.), at 80° C. for 10 minutes to perform a displacement gold plating pretreatment. Subsequently, the metal filter was immersed in HGS-100 at 80° C. for 20 minutes to perform displacement gold plating. A thickness of the displacement gold plating thus obtained was 0.05 μm.

The thus plated metal filter was washed with water, immersed in a non-cyanogen-based reductive electroless gold plating solution, HGS-5400 (manufactured by Hitachi Chemical Co., Ltd.), at 65° C. for 10 minutes to perform gold plating. Thereafter, the metal filter was washed with water and dried. The total thickness of the gold plating thus obtained was 0.2 μm.

(CTC Capturing Apparatus)

Now, a CTC capturing apparatus used in the examples will be described with reference to FIG. 2. The CTC capturing apparatus 100 of FIG. 2 is an apparatus for filtering, with a filter, a subject sample (such as a blood sample) to capture rare cells contained in the sample. Besides, the apparatus can stain the captured rare cells with a stain, identify the rare cells, and count the number of the rare cells.

The CTC capturing apparatus 100 is provided with a filter unit 1 comprising a filter therein, a treatment fluid passage 3 through which a treatment fluid is supplied to the filter unit 1, and a sample passage 4 through which a sample is supplied to the filter unit 1.

On an upstream side of the treatment fluid passage 3, a plurality of treatment fluid containers 5 respectively holding different treatment fluids are provided. Examples of the treatment fluids charged in the treatment fluid containers 5 include a stain to be used for staining rare cells, a fixative, and a washing fluid to be used for washing rare cells captured by a filter. A soft tube is inserted into each of the treatment fluid containers 5 to form an individual treatment fluid passage 6. These passages are connected to a selector valve 8, and the selector valve 8 is rotated to select a treatment fluid to be connected to the treatment fluid passage 3.

A reservoir 10 is connected to the sample passage 4, and the sample is supplied to the reservoir 10. In this structure, either the sample or the treatment fluid is supplied to the filter unit 1, and control for which of the treatment fluid and the sample is to be supplied is performed by switching pinch valves 12 and 13 respectively provided on the passages 3 and 4.

The supply of the treatment fluid and the sample is performed through aspiration with a peristaltic pump 14 provided on a downstream side of the filter unit 1. The sample or the treatment fluid is supplied to the filter unit 1 through the inside of the treatment fluid passage 3 or the sample passage 4, and then flows into a waste fluid tank 16. Rare cells contained in the sample are captured by the filter provided on the passage within the filter unit 1 and stained with a stain.

The above-described components are controlled by a control unit 48. Specifically, the selector valve 8 is controlled by a selector valve driver 49 based on an instruction issued from the control unit 48. The pinch valves 12 and 13 are controlled by two valve drivers 50 respectively connected thereto. The peristaltic pump 14 is driven under control of a pump driver 51.

(Preparation of Cancer Cell Suspension)

NCI-H358 cells, that is, a non-small cell cancer cell line, were statically cultured in a culture flask in RPMI-1640 medium supplemented with 10% fetal bovine serum (FBS) under condition of 5% CO₂ at 37° C. The thus obtained cells were collected by peeling through trypsin treatment, the cells were washed with phosphate buffered saline (PBS) and suspended in PBS containing 2 mM EDTA and 0.5% BSA (hereinafter also referred to as a “washing fluid”) to obtain a suspension of NCI-H358 cells. As the PBS, a product with a product code of 166-23555 manufactured by Wako Pure Chemical Industries Ltd. was used. As the BSA, Albumin from bovine serum-Lyophilized powder, Bio Reagent for cell culture (manufactured by Sigma-Aldrich) was used. As the EDTA-2Na, a product with a product code of 345-01865 manufactured by Wako Pure Chemical Industries Ltd. was used. Subsequently, the concentration of the NCI-H358 cells in the suspension of the NCI-H358 cells was measured using a hemocytometer.

(Preparation of Leukocyte-derived Cells)

Jurkat cells of a leukocyte-derived cell line were statically cultured in a culture flask in RPMI-1640 medium supplemented with 10% fetal bovine serum (FBS) under condition of 5% CO₂ at 37° C. Subsequently, a culture fluid was collected from the culture flask, and the concentration of Jurkat cells therein was measured using a hemocytometer.

### Example 1

The cell suspension of NCI-H358 cells and the cultured Jurkat cells respectively in amounts corresponding to about 1×10³ cancer cells and about 1×10⁵ leukocyte-derived cells were added to the washing fluid to prepare 1 mL of a cell suspension. A sample was prepared by adding, to the cell suspension, 5×10⁶ beads having a diameter of 1 μm (beads obtained by coating polystyrene with PEG300; product code: 01-54-103, trade name: Micromer, manufactured by Micromod).

The above-described CTC capturing apparatus 100 was used for performing the following experiment. First, the reservoir 10 was charged with the washing fluid to fill the filter unit 1 with the washing fluid. Subsequently, the sample was introduced into the reservoir 10, and was fed at a flow rate of 600 μL/min for about 5 minutes. The filter was washed by feeding 3 mL of the washing fluid from the treatment fluid container 5.

A fluid obtained by dissolving 4% PFA, that is, a fixative, in PBS was fed to the filter unit 1, and the filter on which cells had been captured was immersed therein. After washing the filter with the washing fluid, a fluid obtained by dissolving 0.2% Triton X-100 (manufactured by Sigma-Aldrich) in PBS was fed to the filter unit 1, and the filter was immersed therein for 10 minutes.

After washing the filter with the washing fluid, 2 mL of a cell stain (FITC (fluorescein isothiocyanate)-labeled anti-cytokeratin antibody, PE(R-phycoerythrin)-labeled anti-CD45 antibody, 1 μg/mL Hoechst 33342) was fed to the filter unit 1 to fluorescent stain cells on the filter for 30 minutes. Thereafter, the cells were washed with 3 mL of the washing fluid.

The filter was observed with a fluorescence microscope (Axio Imager M2, manufactured by Carl Zeiss Microscopy Co., Ltd.) equipped with a computer-controlled electric stage and a cooled digital camera (AxioCam 506 mono, manufactured by Carl Zeiss Microscopy Co., Ltd.). In order to observe fluorescence derived from FITC, PE and Hoechst 33342, Filter set 38, Filter set 43HE and Filter set 34 (all manufactured by Carl Zeiss Microscopy Co., Ltd.) were respectively used in the fluorescence microscope to obtain images of field of view.

For obtaining and analyzing the images, ZEN (manufactured by Carl Zeiss Microscopy Co., Ltd.) was used. FITC positive cells were regarded as cancer cells and PE positive cells were regarded as leukocytes to count the numbers of these cells, and a purity (%) of captured cancer cells was calculated in accordance with Cancer cell purity=the number of captured cancer cells/the total number of cancer cells and leukocytes captured in Comparative Example 1×100.

### Example 2

An experiment was performed in the same manner as in Example 1 except that the beads to be added to the cell suspension were changed to resin beads having a diameter of 3 μm (product code: 01-54-303, trade name: Micromer, manufactured by Micromod), and a purity of captured cancer cells was determined.

### Example 3

An experiment was performed in the same manner as in Example 1 except that the beads to be added to the cell suspension were changed to resin beads having a diameter of 5 μm (product code: 01-54-503, trade name: Micromer, manufactured by Micromod), and a purity of captured cancer cells was determined.

### Example 4

An experiment was performed in the same manner as in Example 1 except that the beads to be added to the cell suspension were changed to resin beads having a diameter of 7 μm (product code: 01-54-703, trade name: Micromer, manufactured by Micromod), and a purity of captured cancer cells was determined.

### Comparative Example 1

An experiment was performed in the same manner as in Example 1 except that no beads were added to the cell suspension, and a purity of captured cancer cells was determined.

(Evaluation)

Results of the examples and the comparative example are shown in Table 2. In Examples 1 to 4 where the beads were added, the cancer cells could be more selectively captured than in Comparative Example 1 where no beads were added.

TABLE 2

| | Number of | | | | Purity |
|---|---|---|---|---|---|
| | Cancer | Number of | | Number of | of |
| | Cells | Leukocytes | Bead | Beads | Cancer |
| Example | Added | Added | Diameter | Added | Cells |
| Example 1 | 1 × 10³ | 1 × 10⁵ | 1 μm | 5 × 10⁶ | 9.9% |
| Example 2 | 1 × 10³ | 1 × 10⁵ | 3 μm | 5 × 10⁶ | 10.8% |
| Example 3 | 1 × 10³ | 1 × 10⁵ | 5 μm | 5 × 10⁶ | 11.8% |
| Example 4 | 1 × 10³ | 1 × 10⁵ | 7 μm | 5 × 10⁶ | 10.7% |
| Comparative | 1 × 10³ | 1 × 10⁵ | — | — | 7.3% |
| Example 1 | | | | | |

### Example 5

Blood of a healthy person was collected in a blood collection tube (Cell-Free DNA BCT, manufactured by Streck), and after 72 hours had elapsed, a suspension of NCI-H358 cells in an amount corresponding to 1×10³ cancer cells was added thereto to obtain 1 mL of a blood sample. An experiment was performed in the same manner as in Example 1 except that the blood sample was used instead of the cell suspension, that the beads to be added were resin beads having a diameter of 5 μm (product code: 01-54-503, trade name: Micromer, manufactured by Micromod), and that the amount added was set to 1×10⁵ beads, and a purity of captured cancer cells was determined.

### Example 6

An experiment was performed in the same manner as in Example 5 except that the number of the beads added to the blood sample was changed to 1×10⁶, and a purity of captured cancer cells was determined.

### Comparative Example 2

An experiment was performed in the same manner as in Example 5 except that no beads were added to the blood sample, and a purity of captured cancer cells was determined.

(Evaluation)

Results of the examples and the comparative example are shown in Table 3. In Examples 5 to 6 where 1×10⁵ or 1×10⁶ beads were added per 1 mL of the blood sample, the cancer cells could be more selectively captured than in Comparative Example 2 where no beads were added.

TABLE 3

| | Number of | Purity of |
|---|---|---|
| Example | Beads Added | Cancer Cells |
| Example 5 | 1 × 10⁵ | 4.9% |
| Example 6 | 1 × 10⁶ | 6.2% |
| Comparative Example 2 | — | 4.6% |

### REFERENCE SIGNS LIST

1 . . . filter unit, 3, 6 . . . treatment fluid passage, 4 . . . sample passage, 5 . . . treatment fluid container, 8 . . . selector valve, 10 . . . reservoir, 12, 13 . . . pinch valve, 14 . . . peristaltic pump, 16 . . . waste fluid tank, 48 . . . control unit, 49 . . . selector valve driver, 50 . . . valve driver, 51 . . . pump driver, 100 . . . CTC capturing apparatus, 110 . . . through hole, 120 . . . thin film, 200 . . . filter

1. A method for capturing rare cells, comprising:
a step of adding beads to a fluid containing rare cells and contaminating cells; and
a step of filtering, with a filter, the fluid to which the beads have been added.

2. The method according to claim 1, wherein a median diameter of the beads is 0.5 μm to 8 μm.

3. The method according to claim 1, wherein an amount of the beads added is 1×10⁴ to 9×10⁶ beads per 1 mL of the fluid.

4. The method according to claim 1, wherein the beads are at least one type of resin beads and magnetic beads.

5. The method according to claim 1, wherein the contaminating cells are leukocytes.

6. The method according to claim 1, wherein the rare cells are circulating tumor cells or circulating endothelial cells.

7. The method according to claim 2, wherein an amount of the beads added is 1×10⁴ to 9×10⁶ beads per 1 mL of the fluid.

8. The method according to claim 2, wherein the beads are at least one type of resin beads and magnetic beads.

9. The method according to claim 3, wherein the beads are at least one type of resin beads and magnetic beads.

10. The method according to claim 2, wherein the contaminating cells are leukocytes.

11. The method according to claim 3, wherein the contaminating cells are leukocytes.

12. The method according to claim 4, wherein the contaminating cells are leukocytes.

13. The method according to claim 2, wherein the rare cells are blood circulating tumor cells or blood circulating endothelial cells.

14. The method according to claim 3, wherein the rare cells are blood circulating tumor cells or blood circulating endothelial cells.

15. The method according to claim 4, wherein the rare cells are blood circulating tumor cells or blood circulating endothelial cells.

16. The method according to claim 5, wherein the rare cells are blood circulating tumor cells or blood circulating endothelial cells.
